(19) United States
(12) Patent Application Publication
(10) Pub. No.: US 2010/0285130 A1
(43) Pub. Date: Nov. 11, 2010

(54) COATING OF COMPLEXED ACTIVES IN FILM FORMULATIONS

(75) Inventor: Pradeep Sanghvi, Schererville, IN (US)

Correspondence Address: HOFFMANN & BARON, LLP, 6900 JERICHO TURNPIKE, SYOSSET, NY, 11791, US

(73) Assignee: MonoSol Rx, LLC, Portage, IN (US)

(21) Appl. No.: 12/771,611

(22) Filed: Apr. 30, 2010

Related U.S. Application Data
(60) Provisional application No. 61/175,955, filed on May 6, 2009.

(51) Int. Cl. A61K 9/16; A61K 31/439; A61K 31/135

(52) U.S. Cl. 424/484; 514/289; 514/651

(57) ABSTRACT

The present invention relates to products and methods of making products having a dual taste masked active component. In particular, the present invention relates to film dosage forms including at least one dual taste masked active, where the dual taste masked active includes a coated complexed active composition.

### CROSS-REFERENCE TO RELATED APPLICATIONS

The present invention claims priority to U.S. Provisional Application No. 61/175,955, filed May 6, 2009, the entire contents of which are incorporated by reference herein.

### FIELD OF THE INVENTION

The present invention relates to film formulations including at least one active composition. More particularly, the invention relates to products and methods of making products that acceptably taste-mask the active composition.

### BACKGROUND OF THE INVENTION

Administration of active compositions, particularly pharmaceutical compositions, may be achieved in many different forms. Since such active compositions typically have a bitter, foul taste, administration of active compositions may be achieved by swallowing tablets or other dosage forms, which limits the time that the active composition is present in the mouth of the user.

However, it may be desired to administer active compositions through an orally dissolvable film dosage form, which is placed in the mouth and allowed to dissolve, thereby releasing the active composition. Such film dosage forms are beneficial for several reasons, including ease of administration, particularly to those individuals who have difficulty swallowing pills or tablets.

When the film dosage dissolves in the mouth of the user, it exposes taste buds in the mouth to the active composition. Unfortunately, while the active composition is present in the mouth, any portions of the drug which is exposed to the taste buds will result in an unpleasant taste. Methods of taste masking the active composition, while useful, have not been able to sufficiently block the foul taste of actives. Ion exchange resin complexes suffer from premature active release during use or incomplete complexation during manufacture. Simple coating methods often require multiple coatings due to the difficulty in fully coating certain drugs, particularly certain particulate or crystalline shapes.

Moreover, conventional taste masking coatings generally require granulation processing to obtain a usable particle shape and size. There is thus a need for an active component based film composition that effectively masks the foul taste of the active composition and which solves the problems of the prior art, which does not require granulation processing to achieve taste masking effect.

### SUMMARY OF THE INVENTION

In one embodiment of the present invention, there is provided a self-supporting film dosage composition including: a complex of an active and a complexing agent and an ingestible polymer at least partially coating the complex.

In another embodiment, there is provided a method of making a self-supporting film dosage composition including the steps of: complexing an active with a complexing agent to form a complexed active; at least partially coating the complexed active with a polymeric coating to form an at least partially coated complexed active; dispersing a therapeutically effective amount of the coated complexed active into a film-forming polymeric matrix to form an active matrix; and drying the active matrix to form a self-supporting film dosage composition.

Other embodiments of the present invention provide a method of taste masking an active composition, including the steps of: complexing an active with a complexing agent to form a complexed active; and at least partially coating the complexed active with a polymeric coating to form an at least partially coated complexed active.

### DETAILED DESCRIPTION

The present invention provides improved methods and products for oral administration of active components. In some embodiments, the active component (or “drug”) is delivered to the user via an orally dissolvable film strip. The present invention seeks to reduce or altogether eliminate any foul or bitter taste associated with the drug, particularly when administered in a dissolving film dosage.

The present invention provides a pharmaceutical composition in the form of a film for oral administration, including a composition having a uniformly distributed combination of a polymer, a polar solvent, a sweetening agent, and a pharmaceutically active or bioeffecting agent. The composition in its dried film form maintains a uniform distribution of components through the application of controlled bottom drying of the film.

The film dosage composition preferably includes a polymeric carrier matrix, also referred to as a wet film-forming matrix. Any desired polymeric carrier matrix may be used, provided that it is orally dissolvable and is suitable for human ingestion. The orally consumable films are preferably fast-dissolving or moderate-dissolving in the oral cavity and particularly suitable for delivery of actives. However, controlled and sustained release compositions are also among the various embodiments contemplated by the present invention.

The films used in the pharmaceutical products may be produced by a combination of at least one polymer and a polar solvent, optionally including other fillers known in the art. The solvent may be water, a polar organic solvent including, but not limited to, ethanol, isopropanol, acetone, methylene chloride, or any combination thereof. The film may be prepared by utilizing a selected casting or deposition method and a controlled drying process. For example, the film may be prepared through controlled drying processes, which include application of heat and/or radiation energy to the wet film matrix to form a visco-elastic structure in a short period of time (such as less than 10 minutes), thereby controlling the uniformity of content of the film. Such processes are described in more detail in commonly assigned U.S. Pat. Nos. 7,425,292 and 7,357,891, the contents of which are incorporated herein by reference in their entirety. Alternatively, the films may be extruded as described in commonly assigned U.S. Pat. No. 7,666,337, the contents of which are incorporated herein by reference in their entirety.

The polymer may be water soluble, water swellable, water insoluble, or a combination of one or more either water soluble, water swellable or water insoluble polymers. The polymer may include cellulose or a cellulose derivative. Specific examples of useful water soluble polymers include, but are not limited to, polyethylene oxide (PEO), pullulan, hydroxypropylmethyl cellulose (HPMC), hydroxyethyl cellulose (HPC), hydroxypropyl cellulose, polydextrose, polyvinyl pyrrolidone, carboxymethyl cellulose, polyvinyl alcohol, sodium alginate, propylene glycol alginate, carrageenan, polyethylene glycol, xanthan gum, tragancanth gum, guar gum, acacia gum, arabic gum, polyacrylic acid, methylmethacrylate copolymer, poloxamer polymers, copolymers of acrylic acid and alkyl acrylate (availale as Pemulen® polymers), carboxyvinyl copolymers, starch, gelatin, pectin, and combinations thereof.

As used herein the phrase “water soluble polymer” and variants thereof refer to a polymer that is at least partially soluble in water, and desirably fully or predominantly soluble in water, or absorbs water. Polymers that absorb water are often referred to as being water swellable polymers. The materials useful with the present invention may be water soluble or water swellable at room temperature and other temperatures, such as temperatures exceeding room temperature. Moreover, the materials may be water soluble or water swellable at pressures less than atmospheric pressure. Desirably, the water soluble polymers are water soluble or water swellable having at least 20 percent by weight water uptake. Water swellable polymers having a 25 or greater percent by weight water uptake are also useful. Films or dosage forms of the present invention formed from such water soluble polymers are desirably sufficiently water soluble to be dissolvable upon contact with bodily fluids.

Specific examples of useful water insoluble polymers include, but are not limited to, ethyl cellulose, hydroxypropyl ethyl cellulose, cellulose acetate phthalate, hydroxypropyl methyl cellulose phthalate, acrylic polymers, vinyl acetate, sodium sulphonated polyesters, carboxylated acrylics, trimethylpentanediol/adipic acid/glycerin cross polymer, polyglycerol-2-diisostearate/IPDI copolymer, carboxylated vinyl acetate copolymer, vinylpyrrolicone/vinyl acetate/alkylaminoacrylate terpolymers, vinylpyrrolidone/vinyl acetate copolymer, and combinations thereof.

Other polymers useful for incorporation into the films of the present invention include biodegradable polymers, copolymers, block polymers and combinations thereof. Among the known useful polymers or polymer classes which meet the above criteria are: poly(glycolic acid) (PGA), poly(lactic acid) (PLA), polydioxanoes, polyoxalates, poly(α-esters), polyanhydrides, polyacetates, polycaprolactones, poly(orthoesters), polyamino acids, polyaminocarbonates, polyurethanes, polycarbonates, polyamides, poly(alkyl cyanoacrylates), and mixtures and copolymers thereof. Additional useful polymers include, stereopolymers of L- and D-lactic acid, copolymers of bis(p-carboxyphenoxy) propane acid and sebacic acid, sebacic acid copolymers, copolymers of caprolactone, poly(lactic acid)/poly(glycolic acid)/polyethyleneglycol copolymers, copolymers of polyurethane and (poly(lactic acid), copolymers of polyurethane and poly(lactic acid), copolymers of α-amino acids, copolymers of α-amino acids and caproic acid, copolymers of α-benzyl glutamate and polyethylene glycol, copolymers of succinate and poly(glycols), polyphosphazene, polyhydroxy-alkanoates and mixtures thereof. Binary and ternary systems are contemplated.

Other specific polymers useful include those marketed under the Medisorb and Biodel trademarks. The Medisorb materials are marketed by the Dupont Company of Wilmington, Del. and are generically identified as a “lactide/glycolide co-polymer” containing “propanoic acid, 2-hydroxy-polymer with hydroxy-polymer with hydroxyacetic acid.” Four such polymers include lactide/glycolide 100L, believed to be 100% lactide having a melting point within the range of 338°-347° F. (170°-175° C.); lactide/glycolide 100L, believed to be 100% glycolide having a melting point within the range of 437°-455° F. (225°-235° C.); lactide/glycolide 85/15, believed to be 85% lactide and 15% glycolide with a melting point within the range of 338°-347° F. (170°-175° C.); and lactide/glycolide 50/50, believed to be a copolymer of 50% lactide and 50% glycolide with a melting point within the range of 338°-347° F. (170°-175° C.).

The Biodel materials represent a family of various polyanhydrides which differ chemically.

Although a variety of different polymers may be used, it is desired to select polymers to provide a desired viscosity of the mixture prior to drying. For example, if the active or other components are not soluble in the selected solvent, a polymer that will provide a greater viscosity is desired to assist in maintaining uniformity. On the other hand, if the components are soluble in the solvent, a polymer that provides a lower viscosity may be preferred.

The polymer plays an important role in affecting the viscosity of the film. Viscosity is one property of a liquid that controls the stability of the active in an emulsion, a colloid or a suspension. Generally the viscosity of the matrix will vary from about 400 cps to about 100,000 cps, preferably from about 800 cps to about 60,000 cps, and most preferably from about 1,000 cps to about 40,000 cps. Desirably, the viscosity of the film-forming matrix will rapidly increase upon initiation of the drying process.

The viscosity may be adjusted based on the selected active depending on the other components within the matrix. For example, if the component is not soluble within the selected solvent, a proper viscosity may be selected to prevent the component from settling which would adversely affect the uniformity of the resulting film. The viscosity may be adjusted in different ways. To increase viscosity of the film matrix, the polymer may be chosen of a higher molecular weight or crosslinkers may be added, such as salts of calcium, sodium and potassium. The viscosity may also be adjusted by adjusting the temperature or by adding a viscosity increasing component. Components that will increase the viscosity or stabilize the emulsion/suspension include higher molecular weight polymers and polysaccharides and gums, which include without limitation, alginate, carrageenan, hydroxypropyl methyl cellulose, locust bean gum, guar gum, xanthan gum, dextran, gum arabic, gellan gum and combinations thereof.

It has also been observed that certain polymers which when used alone would ordinarily require a plasticizer to achieve a flexible film, can be combined without a plasticizer and yet achieve flexible films. For example, HPMC and HPC when used in combination provide a flexible, strong film with the appropriate plasticity and elasticity for manufacturing and storage. No additional plasticizer or polyalcohol is needed for flexibility.

Additionally, polyethylene oxide (PEO), when used alone or in combination with at least one additional polymer, achieves flexible, strong films. Additional plasticizers or polyalcohols are not needed for flexibility. Non-limiting examples of suitable cellulosic polymers for combination with PEO include HPC and HPMC. PEO and HPC have essentially no gelation temperature, while HPMC has a gelation temperature of 58-64° C. (Methocel EF available from Dow Chemical Co.). Moreover, these films are sufficiently flexible even when substantially free of organic solvents, which may be removed without compromising film properties. As such, if there is no solvent present, then there is no plasticizer in the films. PEO based films also exhibit good resistance to tearing, little or no curling, and fast dissolution rates when the polymer component contains appropriate levels of PEO.

To achieve the desired film properties, the level and/or molecular weight of PEO in the polymer component may be varied. Modifying the PEO content affects properties such as tear resistance, dissolution rate, and adhesion tendencies. Thus, one method for controlling film properties is to modify the PEO content. For instance, in some embodiments rapid dissolving films are desirable. By modifying the content of the polymer component, the desired dissolution characteristics can be achieved.

In accordance with the present invention, PEO desirably ranges from about 20% to 100% by weight in the polymer component. In some embodiments, the amount of PEO desirably ranges from about 1 mg to about 200 mg. The hydrophilic cellulosic polymer ranges from about 0% to about 80% by weight, or in a ratio of up to about 4:1 with the PEO, and desirably in a ratio of about 1:1.

In some embodiments, it may be desirable to vary the PEO levels to promote certain film properties. To obtain films with high tear resistance and fast dissolution rates, levels of about 50% or greater of PEO in the polymer component are desirable. To achieve adhesion prevention, i.e., preventing the film from adhering to the roof of the mouth, PEO levels of about 20% to 75% are desirable. In some embodiments, however, adhesion to the roof of the mouth may be desired, such as for administration to animals or children. In such cases, higher levels of PEO may be employed. More specifically, structural integrity and dissolution of the film can be controlled such that the film can adhere to mucosa and be readily removed, or adhere more firmly and be difficult to remove, depending on the intended use.

The molecular weight of the PEO may also be varied. High molecular weight PEO, such as about 4 million, may be desired to increase mucoadhesivity of the film. More desirably, the molecular weight may range from about 100,000 to 900,000, more desirably from about 100,000 to 600,000, and most desirably from about 100,000 to 300,000. In some embodiments, it may be desirable to combine high molecular weight (600,000 to 900,000) with low molecular weight (100,000 to 300,000) PEOs in the polymer component.

For instance, certain film properties, such as fast dissolution rates and high tear resistance, may be attained by combining small amounts of high molecular weight PEOs with larger amounts of lower molecular weight PEOs. Desirably, such compositions contain about 60% or greater levels of the lower molecular weight PEO in the PEO-blend polymer component.

To balance the properties of adhesion prevention, fast dissolution rate, and good tear resistance, desirable film compositions may include about 50% to 75% low molecular weight PEO, optionally combined with a small amount of a higher molecular weight PEO, with the remainder of the polymer component containing a hydrophilic cellulosic polymer (HPC or HPMC).

In some embodiments, the film may include polyvinyl alcohol (PVA), alone or in combination with at least one additional polymer. Examples of an additional polymer include a cellulosic polymer, starch, polyvinyl pyrrolidone (PVP), polyethylene oxide (PEO), an alginate, a pectin, or combinations thereof. PVA can be used in the films to improve film strength and/or to vary and slow dissolution times. The films are especially useful for the delivery of cosmetics, nutraceuticals and pharmaceuticals. In a preferred embodiment, the film includes PVA without any added platicizers. For example, the film can include both PVA, which provides strength to the film and PEO, which provides flexibility to the film and may obviate the need for a plasticizer.

PVA can be used in varying amounts depending upon the product application and characteristics desired. For example, in general, a larger amount of PVA will increase film strength and increase dissolution time. For films that require high active dosing, PVA can be used effectively at minimum amount of 0.5, preferably 1%, more preferably 5%, by weight of the film, to improve film strength. The PVA can be effectively used at a maximum amount of, for example, 80%, preferably 50%, more preferably 25% by weight of the film. For slowing dissolution time, PVA can be used at levels as high as 80%. A film containing an active can be coated on one or both surfaces with a PVA containing layer to modify the dissolution of the film and the release of an active from the film.

High loading of actives can decrease the strength and flexibility of the film. Including PVA in the film, either alone or in combination with at least one other polymer can increase the tensile strength of the film. Also, drug particles or taste-masked or coated or modified release drug particles may have a larger particle size, which can make loading of these particles into the film difficult. PVA can increase the viscosity of the film solution to allow improved drug loading.

Oral dissolving films generally fall into three main classes: fast dissolving, moderate dissolving and slow dissolving. Fast dissolving films generally dissolve in about 1 second to about 30 seconds. Moderate dissolving films generally dissolve in about 1 to about 30 minutes, and slow dissolving films generally dissolve in more than 30 minutes. Fast dissolving films may consist of low molecular weight hydrophilic polymers (i.e., polymers having a molecular weight between about 1,000 to 9,000). In contrast, slow dissolving films generally have high molecular weight polymers (i.e., having a molecular weight in the millions).

Moderate dissolving films tend to fall in between the fast and slow dissolving films. Moderate dissolving films dissolve rather quickly, but also have a good level of mucoadhesion. Moderate films are also flexible, quickly wettable, and are typically non-irritating to the user. For the instant invention, it is preferable to use films that fall between the categories of fast dissolving and moderate dissolving. Such films provide a quick enough dissolution rate (between about 1 minute and about 5 minutes), while providing an acceptable mucoadhesion level such that the film is not easily removable once it is placed in the oral cavity of the user.

Desirably, the individual film dosage has a small size that is between about 0.5-1 inch by about 0.5-1 inch. Most preferably, the film dosage is about 0.75 inches×0.5 inches. The film dosage should have good adhesion when placed in the buccal cavity or in the sublingual region of the user. Further, the film dosage should disperse and dissolve at a moderate rate, that is, between about 1 minute to about 30 minutes, and most desirably between about 10 minutes and about 20 minutes. In some embodiments, however, it may be desired to allow the individual film dosage to dissolve slower, over a period of longer than about 30 minutes. In such slow dissolving embodiments, it is preferable that the film dosage has strong mucoadhesion properties.

The films of the present invention may include more than one polymer. For instance, in some embodiments, the films may include polyethylene oxide alone or in combination with a second polymer component. In some embodiments, the films may include polymers other than polyethylene oxide. The second polymer may be another water-soluble polymer, a water-swellable polymer, a water-insoluble polymer, a biodegradable polymer or any combination thereof Suitable water-soluble polymers include, without limitation, any of those provided above.

In accordance with some embodiments, polyethylene oxide may range from about 20% to 100% by weight in the polymer component, more specifically about 30% to about 70% by weight, and even more specifically about 40% to about 60% by weight. In some embodiments, one or more water-swellable, water-insoluble and/or biodegradable polymers also may be included in the polyethylene oxide-based film. Any of the water-swellable, water-insoluble or biodegradable polymers provided above may be employed. The second polymer component may be employed in amounts of about 0% to about 80% by weight in the polymer component, more specifically about 30% to about 70% by weight, and even more specifically about 40% to about 60% by weight.

The molecular weight of the polyethylene oxide also may be varied. In some embodiments, high molecular weight polyethylene oxide, such as about 4 million, may be desired to increase mucoadhesivity of the film. In some other embodiments, the molecular weight may range from about 100,000 to 900,000, more specifically from about 100,000 to 600,000, and even more specifically from about 100,000 to 300,000. In some embodiments, it may be desirable to combine high molecular weight (600,000 to 900,000) with low molecular weight (100,000 to 300,000) polyethylene oxide in the polymer component.

A variety of optional components and fillers also may be added to the films. These may include, without limitation: surfactants; plasticizers; polyalcohols; anti-foaming agents, such as silicone-containing compounds, which promote a smoother film surface by releasing oxygen from the film; thermo-setting gels such as pectin, carageenan, and gelatin, which help in maintaining the dispersion of components; inclusion compounds, such as cyclodextrins and caged molecules; coloring agents; and flavors. In some embodiments, more than one active ingredient may be included in the film.

Additives may be included in the films. Examples of classes of additives include excipients, lubricants, buffering agents, stabilizers, blowing agents, pigments, coloring agents, fillers, bulking agents, sweetening agents, flavoring agents, fragrances, release modifiers, adjuvants, plasticizers, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers and mixtures thereof. These additives may be added with the active agent(s).

Useful additives include, for example, gelatin, vegetable proteins such as sunflower protein, soybean proteins, cotton seed proteins, peanut proteins, grape seed proteins, whey proteins, whey protein isolates, blood proteins, egg proteins, acrylated proteins, water-soluble polysaccharides such as alginates, carrageenans, guar gum, agar-agar, xanthan gum, gellan gum, gum arabic and related gums (gum ghatti, gum karaya, gum tragancanth), pectin, water-soluble derivatives of cellulose: alkylcelluloses hydroxyalkylcelluloses and hydroxyalkylalkylcelluloses, such as methylcelulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate (CAP), hydroxypropylmethylcellulose (HPMC); carboxyalkylcelluloses, carboxyalkylalkylcelluloses, carboxyalkylcellulose esters such as carboxymethylcellulose and their alkali metal salts; water-soluble synthetic polymers such as polyacrylic acids and polyacrylic acid esters, polymethacrylic acids and polymethacrylic acid esters, polyvinylacetates, polyvinylalcohols, polyvinylacetatephthalates (PVAP), polyvinylpyrrolidone (PVP), PVY/vinyl acetate copolymer, and polycrotonic acids; also suitable are phthalated gelatin, gelatin succinate, crosslinked gelatin, shellac, water-soluble chemical derivatives of starch, cationically modified acrylates and methacrylates possessing, for example, a tertiary or quaternary amino group, such as the diethylaminoethyl group, which may be quaternized if desired; and other similar polymers.

Such extenders may optionally be added in any desired amount desirably within the range of up to about 80%, desirably about 3% to 50% and more desirably within the range of 3% to 20% based on the weight of all film components.

Further additives may be inorganic fillers, such as the oxides of magnesium aluminum, silicon, titanium, etc. desirably in a concentration range of about 0.02% to about 3% by weight and desirably about 0.02% to about 1% based on the weight of all film components.

Further examples of additives are plasticizers which include polyalkylene oxides, such as polyethylene glycols, polypropylene glycols, polyethylene-propylene glycols, organic plasticizers with low molecular weights, such as glycerol, glycerol monoacetate, diacetate or triacetate, triacetin, polysorbate, cetyl alcohol, propylene glycol, sorbitol, sodium diethylsulfosuccinate, triethyl citrate, tributyl citrate, and the like, added in concentrations ranging from about 0.5% to about 30%, and desirably ranging from about 0.5% to about 20% based on the weight of the polymer.

There may further be added compounds to improve the flow properties of the starch material such as animal or vegetable fats, desirably in their hydrogenated form, especially those which are solid at room temperature. These fats desirably have a melting point of 50° C. or higher. Preferred are tri-glycerides with C₁₂-, C₁₄-, C₁₆-, C₁₈-, C₂₀- and C₂₂-fatty acids. These fats can be added alone without adding extenders or plasticizers and can be advantageously added alone or together with mono- and/or di-glycerides or phosphatides, especially lecithin. The mono- and di-glycerides are desirably derived from the types of fats described above, i.e. with C₁₂-, C₁₄-, C₁₆-, C₁₈-, C₂₀- and C₂₂-fatty acids.

The total amounts used of the fats, mono-, di-glycerides and/or lecithins may be up to about 5% and preferably within the range of about 0.5% to about 2% by weight of the total film composition.

It further may be useful to add silicon dioxide, calcium silicate, or titanium dioxide in a concentration of about 0.02% to about 1% by weight of the total composition. These compounds act as texturizing agents.

Lecithin is one surface active agent for use in the films described herein. Lecithin may be included in the feedstock in an amount of from about 0.25% to about 2.00% by weight. Other surface active agents, i.e. surfactants, include, but are not limited to, cetyl alcohol, sodium lauryl sulfate, the Spans™ and Tweens™ which are commercially available from ICI Americas, Inc. Ethoxylated oils, including ethoxylated castor oils, such as Cremophor® EL which is commercially available from BASF, are also useful. Carbowax™ is yet another modifier which is very useful in the present invention. Tweens™ or combinations of surface active agents may be used to achieve the desired hydrophilic-lipophilic balance (“HLB”).

Other ingredients include binders which contribute to the ease of formation and general quality of the films. Non-limiting examples of binders include starches, pregelatinize starches, gelatin, polyvinylpyrrolidone, methylcellulose, sodium carboxymethylcellulose, ethylcellulose, polyacrylamides, polyvinyloxoazolidone, and polyvinylalcohols. If desired, the film may include other additives, such as keratin, or proteins, including proteins that are useful in forming a gel, such as gelatine.

Further potential additives include solubility enhancing agents, such as substances that form inclusion compounds with active ingredients. Such agents may be useful in improving the properties of very insoluble and/or unstable actives. In general, these substances are doughnut-shaped molecules with hydrophobic internal cavities and hydrophilic exteriors. Insoluble and/or instable actives may fit within the hydrophobic cavity, thereby producing an inclusion complex, which is soluble in water. Accordingly, the formation of the inclusion complex permits very insoluble and/or instable actives to be dissolved in water. A particularly desirable example of such agents are cyclodextrins, which are cyclic carbohydrates derived from starch. Other similar substances, however, are considered well within the scope of the present invention.

Suitable coloring agents include food, drug and cosmetic colors (FD&C), drug and cosmetic colors (D&C), or external drug and cosmetic colors (Ext. D&C). These colors are dyes, their corresponding lakes, and certain natural and derived colorants. Lakes are dyes absorbed on aluminum hydroxide.

Other examples of coloring agents include known azo dyes, organic or inorganic pigments, or coloring agents of natural origin. Inorganic pigments are preferred, such as the oxides or iron or titanium, these oxides, being added in concentrations ranging from about 0.001 to about 10%, and preferably about 0.5 to about 3%, based on the weight of all the components.

Flavors may be chosen from natural and synthetic flavoring liquids. An illustrative list of such agents includes volatile oils, synthetic flavor oils, flavoring aromatics, oils, liquids, oleoresins or extracts derived from plants, leaves, flowers, fruits, stems and combinations thereof. A non-limiting representative list of examples includes mint oils, cocoa, and citrus oils such as lemon, orange, grape, lime and grapefruit and fruit essences including apple, pear, peach, grape, strawberry, raspberry, cherry, plum, pineapple, apricot or other fruit flavors.

Other useful flavorings include aldehydes and esters such as benzaldehyde (cherry, almond), citral i.e., alphacitral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanol (green fruit), and 2-dodecenal (citrus, mandarin), combinations thereof and the like.

The sweeteners may be chosen from the following non-limiting list: glucose (corn syrup), dextrose, invert sugar, fructose, and combinations thereof; saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; sugar alcohols such as sorbitol, mannitol, xylitol, and the like. Also contemplated are hydrogenated starch hydrolysates and the synthetic sweetener 3,6-dihydro-6-methyl-1-1-1,2,3-oxathiazin-4-one-2,2-dioxide, particularly the potassium salt (acesulfame-K), and sodium and calcium salts thereof, and natural intensive sweeteners, such as Lo Han Kuo. Other sweeteners may also be used.

The films may include one or more additives to provide a taste masking of the active ingredient. For example, the films may include ionic exchange resins, including but not limited to a water-insoluble organic or inorganic matrix material having covalently bound functional groups that are ionic or capable of being ionized under appropriate conditions. The organic matrix may be synthetic (e.g., polymers or copolymers or acrylic acid, methacrylic acid, sulfonated styrene or sulfonated divinylbenzene) or partially synthetic (e.g., modified cellulose or dextrans). The inorganic matrix may be, for example, silica gel modified by the addition of ionic groups. Most ion exchange resins are cross-linked by a crosslinking agent, such as divinylbenzene.

Anti-foaming and/or de-foaming components may also be used with the films. These components aid in the removal of air, such as entrapped air, from the film-forming compositions. Such entrapped air may lead to non-uniform films. Simethicone is one particularly useful anti-foaming and/or de-foaming agent. The present invention, however, is not so limited and other anti-foam and/or de-foaming agents may suitable be used.

As a related matter, simethicone and related agents may be employed for densification purposes. More specifically, such agents may facilitate the removal of voids, air, moisture, and similar undesired components, thereby providing denser, and thus more uniform films. Agents or components which perform this function can be referred to as densification or densifying agents. As described above, entrapped air or undesired components may lead to non-uniform films.

Simethicone is generally used in the medical field as a treatment for gas or colic in babies. Simethicone is a mixture of fully methylated linear siloxane polymers containing repeating units of polydimethylsiloxane which is stabilized with trimethylsiloxy end-blocking unites, and silicon dioxide. It usually contains 90.5-99% polymethylsiloxane and 4-7% silicon dioxide. The mixture is a gray, translucent, viscous fluid which is insoluble in water.

When dispersed in water, simethicone will spread across the surface, forming a thin film of low surface tension. In this way, simethicone reduces the surface tension of bubbles air located in the solution, such as foam bubbles, causing their collapse. The function of simethicone mimics the dual action of oil and alcohol in water. For example, in an oily solution any trapped air bubbles will ascend to the surface and dissipate more quickly and easily, because an oily liquid has a lighter density compared to a water solution. On the other hand, an alcohol/water mixture is known to lower water density as well as lower the water's surface tension. So, any air bubbles trapped inside this mixture solution will also be easily dissipated. Simethicone solution provides both of these advantages. It lowers the surface energy of any air bubbles that trapped inside the aqueous solution, as well as lowering the surface tension of the aqueous solution. As the result of this unique functionality, simethicone has an excellent anti-foaming property that can be used for physiological processes (anti-gas in stomach) as well as any for external processes that require the removal of air bubbles from a product.

In order to prevent the formation of air bubbles in the films, the mixing step may be performed under vacuum. However, as soon as the mixing step is completed, and the film solution is returned to the normal atmosphere condition, air will be re-introduced into or contacted with the mixture. In many cases, tiny air bubbles will be again trapped inside this polymeric viscous solution. The incorporation of simethicone into the film-forming composition either substantially reduces or eliminates the formation of air bubbles during and after mixing.

Simethicone may be added to the film-forming mixture as an anti-foaming agent in an amount from about 0.01 weight percent to about 5.0 weight percent, more desirably from about 0.05 weight percent to about 2.5 weight percent, and most desirably from about 0.1 weight percent to about 1.0 weight percent.

Any other optional components described in commonly assigned U.S. Pat. Nos. 7,425,292 and 7,357,891 and U.S. application Ser. No. 10/856,176, referred to above, also may be included in the films described herein.

The active component or components in the film composition are desirably taste-masked, so as to prevent the user from the foul or bitter taste of the active. Further, since the film composition will remain in the mouth for an extended period of time (i.e., at least 30 seconds or one minute), it is important that the active be sufficiently taste-masked for the time that the active is in the mouth. In addition, it is important that substantially all of the active in the film composition be effectively taste-masked to avoid any bitter taste perception. For these reasons, it is desired that the active(s) be subjected to a dual-masking process.

The active(s) in the present invention are subjected to a dual-masking process, where the active is first complexed with an ion exchange resin, and then the complexed active is coated with a suitable and ingestible coating. In this manner, the likelihood that any active will be either unbound or uncoated is extremely low, thereby reducing the potential for loose active in the film and thus a foul taste perception by the user. The Applicant has found that systems which incorporate only a method of binding the active with an ion exchange resin are insufficient for several reasons. First, there is a likelihood that a portion of the active will be uncomplexed with the resin, thereby leaving some unbound (and thus un-taste-masked) actives in the film. In addition, even bound actives may have a tendency to become dissociated while the film is in the mouth of the user, thus creating a foul taste perception to the user. Further, the Applicant has found that systems which incorporate only a coated active are likewise insufficient to adequately reduce foul taste perception. As with systems which only incorporate binding the active, there is a high likelihood that not all of the active will be coated prior to delivery into the film. Thus, the film includes a portion of uncoated (and thus un-taste-masked) active. When this film is placed into the oral cavity of the user, the uncoated active is released and provides a foul taste to the user.

To avoid these potential problems, the active or actives to be dispensed in the film are dual taste masked. The present method of dual taste masking can be applied to any active ingredient desired. Without limitation, exemplary actives that can be used in the present method include ace-inhibitors, antianginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, biological response modifiers, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, terine relaxants, anti-obesity drugs, erythropoietic drugs, anti-asthmatics, cough suppressants, mucolytics, DNA and genetic modifying drugs, and combinations thereof.

Examples of medicating active ingredients contemplated for use in the present invention include antacids, H₂-antagonists, and analgesics. For example, antacid dosages can be prepared using the ingredients calcium carbonate alone or in combination with magnesium hydroxide, and/or aluminum hydroxide. Moreover, antacids can be used in combination with H₂-antagonists.

Analgesics include opiates and opiate derivatives, such as oxycodone (available as Oxycontin®), ibuprofen, aspirin, acetaminophen, and combinations thereof that may optionally include caffeine.

Other preferred drugs for other preferred active ingredients for use in the present invention include anti-diarrheals such as immodium AD, anti-histamines, anti-tussives, decongestants, vitamins, and breath fresheners. Common drugs used alone or in combination for colds, pain, fever, cough, congestion, runny nose and allergies, such as acetaminophen, chlorpheniramine maleate, dextromethorphan, pseudoephedrine HCl and diphenhydramine may be included in the film compositions of the present invention.

Also contemplated for use herein are anxiolytics such as alprazolam (available as Xanax®); anti-psychotics such as clozopin (available as Clozaril®) and haloperidol (available as Haldol®); non-steroidal anti-inflammatories (NSAID's) such as dicyclofenacs (available as Voltaren®) and etodolac (available as Lodine®), anti-histamines such as loratadine (available as Claritin®), astemizole (available as Hismanal™), nabumetone (available as Relafen®), and Clemastine (available as Tavist®); anti-emetics such as granisetron hydrochloride (available as Kytril®), serotonin 5-HT3 receptor antagonists (available as Ondansetron) and nabilone (available as Cesamet™); bronchodilators such as Bentolin®, albuterol sulfate (available as Proventil®); anti-depressants such as fluoxetine hydrochloride (available as Prozac®), sertraline hydrochloride (available as Zoloft®), and paroxtine hydrochloride (available as Paxil®); anti-migraines such as Imigra®, ACE-inhibitors such as enalaprilat (available as Vasotec®), captopril (available as Capoten®) and lisinopril (available as Zestril®); anti-Alzheimer's agents, such as nicergoline; and Ca^{H}-antagonists such as nifedipine (available as Procardia® and Adalat®), and verapamil hydrochloride (available as Calan®).

Erectile dysfunction therapies include, but are not limited to, drugs for facilitating blood flow to the penis, and for effecting autonomic nervous activities, such as increasing parasympathetic (cholinergic) and decreasing sympathetic (adrenersic) activities. Useful non-limiting drugs include sildenafils, such as Viagra®, tadalafils, such as Cialis®, vardenafils, apomorphines, such as Uprima®, yohimbine hydrochlorides such as Aphrodyne®, and alprostadils such as Caverject®.

The popular H₂-antagonists which are contemplated for use in the present invention include cimetidine, ranitidine hydrochloride, famotidine, nizatidien, ebrotidine, mifentidine, roxatidine, pisatidine and aceroxatidine.

Active antacid ingredients include, but are not limited to, the following: aluminum hydroxide, dihydroxyaluminum aminoacetate, aminoacetic acid, aluminum phosphate, dihydroxyaluminum sodium carbonate, bicarbonate, bismuth aluminate, bismuth carbonate, bismuth subcarbonate, bismuth subgallate, bismuth subnitrate, bismuth subsilysilate, calcium carbonate, calcium phosphate, citrate ion (acid or salt), amino acetic acid, hydrate magnesium aluminate sulfate, magaldrate, magnesium aluminosilicate, magnesium carbonate, magnesium glycinate, magnesium hydroxide, magnesium oxide, magnesium trisilicate, milk solids, aluminum mono-ordibasic calcium phosphate, tricalcium phosphate, potassium bicarbonate, sodium tartrate, sodium bicarbonate, magnesium aluminosilicates, tartaric acids and salts.

The pharmaceutically active agents employed in the present invention may include allergens or antigens, such as , but not limited to, plant pollens from grasses, trees, or ragweed; animal danders, which are tiny scales shed from the skin and hair of cats and other furred animals; insects, such as house dust mites, bees, and wasps; and drugs, such as penicillin.

In a method of dual taste masking the active components, the active component or components are first associated with a complexing agent to form a complexed active. In some embodiments, the complexing agent includes at least one ion exchange resin so as to form the complexed active component. Any ion exchange resin or resins may be used in the present method. Ion exchange resins may serve several different functions in pharmaceutical applications, including extended- or controlled-release, taste-masking, and improving the stability of actives. Ion exchange resins generally are insoluble macromolecules or polyelectrolytes that have electrically charged sites at which one ion may replace another ion. Cation-exchange resins have fixed electronegative charges that interact with counterions having the opposite, or positive, charge. Cation-exchange resins exchange positively charged cations. Anion-exchange resins have electropositive charges that interact with counterions having the opposite, or negative, charge. Anion-exchange resins exchange negatively charged anions.

Without limitation, exemplary ion exchange resins include water-insoluble organic or inorganic matrix materials having covalently bound functional groups that are ionic or capable of being ionized under appropriate conditions. The organic matrix may be synthetic (e.g., polymers or copolymers or acrylic acid, methacrylic acid, sulfonated styrene or sulfonated divinylbenzene) or partially synthetic (e.g., modified cellulose or dextrans). The inorganic matrix may be, for example, silica gel modified by the addition of ionic groups. Many ion exchange resins are cross-linked by a crosslinking agent, such as divinylbenzene.

Ion exchange resins for use herein may be categorized into four main types depending on their functional groups: strongly acidic (e.g., sulfonic acid groups); strongly basic (e.g., trimethylammonium groups); weakly acidic (e.g., carboxylic acid groups); and weakly basic (e.g., amino groups).

In some embodiments, for instance, an acidic resin may be employed. The acidic resin may be combined with a basic drug to form a complexate. Examples of acidic resins that can be combined with basic drugs include, but are not limited to, partially neutralized poly(acrylic acid), crosslinked acrylic acid copolymers (such as Indion 414), sodium polystyrene sulfonate (such as Amberlite IRP-69), copolymers of methyacrylic acid crosslinked with divinylbenzene (such as Amberlite IRP-64), and polacrilin potassium.

Examples of basic drugs that can be combined with any of the acidic resins set forth above include, but are not limited to, levobetaxolol hyrdrochloride, roxithromycin, dicyclomine hydrochloride, montelukast sodium, dextromethorphan hydrobromide, diphenhydramine hydrochloride, orbifloxacin, ciprofloxacin, enoxacin, grepafloxacin, levofloxacin, lomefloxacin, nalidixic acid, acycloguanosine, tinidazole, deferiprone, cimetidine, oxycodone, remacemide, nicotine, morphine, hydrocodone, rivastigmine, propanolol, betaxolol, chlorpheniramine, and paroxetine.

In some embodiments, a basic resin may be employed. The basic resin may be combined with an acidic drug to form a complexate. Examples of basic resins that can be used to form complexates include, but are not limited to, polyvinylpyrrolidone, polylysine, polyarginine, and polyhistidine.

Examples of acidic drugs that can be combined with any of the basic resins set forth above include, but are not limited to, nicotinic acid, mefanamic acid, indomethacin, diclofenac, repaglinide, ketoprofen, ibuprofen, valproic acid, lansoprazole, ambroxol, omeprazole, acetaminophen, topiramate, amphotericin B, and carbemazepime.

In some other embodiments, the complexing agent used to bond to the active may rely on weak bonding forces, such as Van der Waals forces or hydrogen bonding, to form a complexate with an initial active. Such complexing agents may include caged molecules, such as cyclodextrins. Cyclodextrins generally are cyclic oligosaccharides composed of alpha-D-glucopyranose units. Common cyclodextrins include alpha-, beta- and gamma-cyclodextrins, which contain 6, 7 and 8 glucose units, respectively. Cyclodextrins have a toroidal shape with a generally hydrophobic interior cavity and a generally hydrophilic exterior, which imparts water-solubility to the molecule. This characteristic allows cyclodextrins to form inclusion complexes, i.e., host-guest complexes, with hydrophobic molecules to increase the water-solubility thereof. More specifically, guest molecules interact with the interior cavity of the cyclodextrin to become entrapped and form a stable association therewith. Due to the hydrophilic exterior of the cyclodextrin, the inclusion complex is water-soluble, thereby increasing the release of poorly soluble drugs complexed therewith.

Examples of such complexing agents include, but are not limited to, alpha-cyclodextrin, beta-cyclodextrin, gamma-cyclodextrin and derivatives of cyclodextrins, such as hydroxyalkylated cyclodextrins. Examples of drugs that can be combined with this type of complexing agent are many and are determined by the fit of the drug within the complexing agent, e.g., cyclodextrin. For example, anthracyclines form good complexes with gamma-cyclodextrin. Complexes of other cyclodextrins are described in U.S. Pat. No. 4,727,064, which is incorporated herein by reference in its entirety.

In some embodiments described herein, the complexing agent may be a zeolite. Zeolites are minerals having a micro-porous structure. Zeolites include naturally occurring minerals and synthetic compounds, which generally are characterized by an alumino-silicate framework with an open structure that can accommodate cations, such as Na⁺, K⁺, Ca²⁺, Sr²⁺ and Ba²⁺. The cations reside in cavities in the crystal structure and can be readily exchanged for others in a solution. Zeolites can be of various different types, such as P-type and X-type, and with numerous counterions, such as sodium and calcium. Additionally, zeolites can be used in combination with ammonium salts, such as hexadecyltrimethyl ammonium bromide. An example of this is a complex of chloroquin with a P-type zeolite with a sodium counterion and in the presence of dodecyltrimethylammonium bromide.

In some embodiments, the complexing agent may rely on any type of molecular entanglement, as such entanglement is understood in quantum theory. Any materials that are bound in any way are by definition “entangled” in quantum theory.

In such embodiments, the molecular chains of a complexing agent, such as a polymer, are sufficiently entangled to trap or bind the active, thereby forming the complexate. In instances when the molecular weight is excessive, the ability of the thus formed complexate to release the active may be hampered or too slow for practical purposes. Thus, the upper limit for molecular weight of the complexing agent is that which still provides efficacy for its intended use. The upper limits of molecular weight will of course depend on the polymer chosen, as well as the active, since the behavior of the complexate is dependent to a large degree on its formative components.

Once the active component(s) is bound to the complexing agent, such as an ion exchange resin, the complexed active may then be coated with an ingestible coating to form coated active(s). The coating is desirably a polymeric coating, and most desirably is a polymeric coating that is water-insoluble at a neutral pH. The coating preferably is capable of breaking down in an acidic pH environment, such as the gastric region of the body. The coating should be capable of sustaining its composition while in the oral cavity of the user, in the presence of the approximately neutral pH of the saliva of the user. Once ingested, the coated active will be in the presence of gastric acid, where the coating is capable of breaking down to release the bound active component or components.

The taste-masking coating may therefore prevent or at least minimize the release of the drug from the coating both during the manufacturing process of the oral thin film, as well as when the film is administered in the oral cavity of the consumer. As explained above, the film may be present in the oral cavity for an extended period of time, such as at least thirty seconds or even one minute or longer, during which the coated active is released from the film. The coating is preferably strong enough to prevent release of the active while the coated active is in the oral cavity, thereby minimizing potential for a foul taste perception to the user.

Any suitable coating materials can be used, provided that the coating materials are ingestible and fit for human consumption. For example, particles of a drug may be coated with polymers, such as ethyl cellulose or polymethacrylate, which are commercially available under brand names such as Aquacoat ECD and Eudragit E-100 as sold by Evonik Industries, respectively. Without limitation, coating materials may include a reverse-enteric polymer. Examples of reverse-enteric polymers may include copolymers of dimethyl aminoethyl methacrylate and neutral methacrylic acid esters, and water-insoluble, pH independent base polymeric constituent, such as cellulose acetate or ethylcellulose.

In some embodiments, excipients may be added to the coating composition to further increase the rate of release of the drug from the film. Desirably, the taste-masking properties are still maintained after the addition of these excipients. One example of an useful excipients for use in the oral thin film is an acid reactive material, such as calcium carbonate or calcium phosphate. Alternatively, any related or unrelated materials that may react with stomach acid may be used.

The coated and bound active is preferably in the form of coated particles, which may then be dispersed throughout the polymeric film-forming matrix. The coated particles may be any size desired so as to provide an even dispersion and allow the matrix to form a film. In some embodiments, the coated actives have a particle size of about 10 to about 200 microns. Desirably, the size of the coated particle may be a particle size of 150 microns or less, for example 100 microns or less. Moreover, such particles may be spherical, substantially spherical, or non-spherical, such as irregularly shaped particles or ellipsoidally shaped particles. Ellipsoidally shaped particles or ellipsoids are desirable because of their ability to maintain uniformity in the film forming matrix as they tend to settle to a lesser degree as compared to spherical particles. It may be desired, however, to incorporate larger sized coated particles, such coated particles having a particle size of greater than 200 microns.

The resulting dual taste masked composition thus includes a core of the bound (or complexed) active component with a polymeric coating shell. The core may include one bound active or it may include more than one bound active.

A method of forming a film dosage incorporating at least one dual taste masked active is provided herein. The film composition includes an amount of the dual taste masked active and a polymeric film-forming matrix, as described above. The matrix may include any number of additional components such as set forth above, including flavors, colors, sweeteners, other taste-masking agents, and other additives as desired. In one method of forming the film of the present invention, the active is first bound to a complexing agent, such as an ion exchange resin. In some embodiments, the active and ion exchange resin are combined in an equal ratio, although it may be desired to incorporate more ion exchange resin than active component to ensure that substantially all of the active component is bound to the complexing agent. The active may be present in amounts of about 0.01% to about 60% by weight of the complexing agent. In some embodiments, the active may be present in amounts of from about 0.1% to about 20% by weight of the complexing agent.

Once the active component is bound to the complexing agent, the complexed active may then be coated with a coating material as set forth above. Any desired means of coating may be employed, including such as spraying or other means. In some embodiments, the coating may be applied from an organic solution, such as acetone, on to the complexed drug particles or granules in a fluid bed dryer using a top-spray, bottom-spray or Wurster column bottom spray configuration.

Once the desired active or actives have been complexed and coated to form a dual taste masked product, the dual taste masked product may be incorporated into the film forming polymeric matrix. The film-forming polymeric matrix may then be used to form the self-supporting film dosage form via any known method of forming films, such as those described in U.S. Pat. Nos. 7,425,292 and 7,357,891, the entire contents of which are incorporated by reference herein.

### EXAMPLES

### Example 1

### Bound and Coated Dextromethorphan

Dextromethorphan HBr is bound with a polystyrene-divinylbenzene to form a complex. The complexed resinate is then coated with Eudragit E100, a methacrylate copolymer. The Eudragit E100 provides a reverse enteric coating. The coated and complexed active is swellable and dissolvable in a gastric pH, thereby allowing release of the active from the coating. Once released from the coating, the active dissociates from the complex and may be ingested into the system. However, the Eudragit E100 coating is not dissolvable in a salivary pH, thereby preventing premature release of the complexed active while in the oral cavity of the user. Since the coated and complexed active is not dissolvable at salivary pH, the bitter taste of the active is avoided.

### Example 2

### Bound and Coated Diphenhydramine

Diphenhydramine HCl is bound with sodium polystyrene sulfonate to form a complex. The complex is then coated with hypromellose. This coating is capable of dissolving in a gastric pH, allowing the release of the drug from the coating. Once released from the coating, the drug is allowed to dissociate from the complex and is released into the body.

### Example 3

### Bound and Coated Propranolol

Propranolol HCl is bound with Amberlite IRP69 (Sodium Polystyrene Sulfonate) to form a complex. The complex is then coated with an ethylcellulose:HPC (80:20) coating to form the coated complexed drug. This coated complexed drug is capable of dissolving in a gastric pH, allowing the release of the drug from the coating once swallowed.

What is claimed is:

1. A self-supporting film dosage composition comprising: a complex of an active and a complexing agent; and an ingestible polymer at least partially coating said complex.

2. The composition of claim 1, wherein said complexing agent comprises an ion exchange resin.

3. The composition of claim 1, wherein said coated complexed active comprises a particle.

4. The composition of claim 3, wherein said particle has a particle size of about 10 to about 200 microns.

5. A method of making a self-supporting film dosage composition comprising the steps of:
a. complexing an active with a complexing agent to form a complexed active;
b. at least partially coating said complexed active with a polymeric coating to form an at least partially coated complexed active;
c. dispersing a therapeutically effective amount of said coated complexed active into a film-forming polymeric matrix to form an active matrix; and
d. drying said active matrix to form a self-supporting film dosage composition.

6. The method of claim 5, wherein said complexing agent comprises an ion exchange resin.

7. The method of claim 5, wherein said coated complexed active comprises a particle.

8. The method of claim 7, wherein said particle has a particle size of about 10 to about 200 microns.

9. A method of taste masking an active composition, comprising the steps of:
a. complexing an active with a complexing agent to form a complexed active; and
b. at least partially coating said complexed active with a polymeric coating to form an at least partially coated complexed active.

10. The method of claim 9, wherein said complexing agent comprises an ion exchange resin.

11. The method of claim 9, wherein said coated complexed active comprises a particle.

12. The method of claim 11, wherein said particle has a particle size of about 10 to about 200 microns.
